(19) United States
(12) Patent Application Publication
(10) Pub. No.: US 2022/0175237 A1
(43) Pub. Date: Jun. 9, 2022

(54) STRUCTURAL ARRANGEMENT APPLIED TO A RIGID ENDOSCOPE FOR CLEANING THE OBJECTIVE LENS DURING VIDEO SURGERY PROCEDURE

(71) Applicant: Igor Renato De Abreu, Sao Paulo (BR)

(72) Inventor: Igor Renato De Abreu, Sao Paulo (BR)

(21) Appl. No.: 17/599,233

(22) PCT Filed: Jan. 30, 2020
(86) PCT No.: PCT/BR2020/050019

§ 371 Date: Sep. 28, 2021

(51) Int. Cl. A61B 1/12; A61B 1/015

(52) U.S. Cl. CPC A61B 1/126; A61B 1/015

(57) ABSTRACT

The present utility model is related to the medical field, and more specifically to a new arrangement applied to rigid endoscopes (1), which comprises the arrangement of two channels superior upper (3) and lower (4) longitudinally arranged on the rigid shaft (11) of the endoscope (1), with the upper channel (3) receiving a piece of equipment (5) on its proximal end capable of releasing, in a controlled way, an amount of crystalloid solution, which is directed by gravity to the distal end (12), lubricating the objective lens of the endoscope, and with the lower channel (4) connected to suction device (7), through a hose (43) with valve (8), which when activated, sucks the saline, cleaning the lens (2) during the video-surgery procedure, removing blood or body secretions, and improving the visibility of this procedure for the surgeon.

The present utility model is related to the medical field, and more specifically, to a new arrangement applied to rigid endoscopes. Such arrangement comprises two channels (upper and lower) longitudinally arranged on the rigid shaft of the endoscope, with the upper channel receiving a piece of equipment on its fore end capable of releasing, in a controlled way, an amount of crystalloid solution, which is directed by gravity to the distal end, lubricating the objective lens of the endoscope, and with the lower channel connected to suction device and receiving on its fore end a valve, which when activated, sucks the solution and cleans the lens during the video-surgery procedure, removing blood or body secretions, and improving the visibility of this procedure for the surgeon.

### FOUNDATIONS OF THE TECHNIQUE

The current technological advancements in all the human knowledge areas, and the application of this knowledge in the health areas, enabled the development of minimally invasive surgical treatment techniques. Undoubtedly, a crucial issue in this achievement was the introduction of video surgeries and robotic surgeries.

It is crystal clear that to perform a surgical procedure, the surgeon must have good vision of what is going on, and this is achieved by good surgical optics.

There are several sizes of endoscopes with optical features in several angulations of objective lenses.

However, during a surgical procedure by video, the objective lens gets frequently stained with blood or body secretions of the patient, blurring the surgeon's vision. When this occurs, it is necessary to remove the endoscope from inside the patient to be cleaned, but this maneuver interrupts the surgical act for nearly 20 seconds.

It must be considered that in some surgical procedures, under no circumstances, should the surgeon miss the vision of what is going on inside the patient.

However, in some surgeries, large amounts of secretions and blood are handled, staining the optical pieces several times, and impairing the surgical time.

### STATE OF THE ART

The state of the art document PI9201776-2, filed on May 5, 1992, discloses a device for cleaning distal lenses of optical instruments, such as rigid endoscopes, which are internally or externally provided a channel with microtube shape, through which it is possible to inject saline by syringe or pressurizing pump, as intermittent jet sprays directed against the objective lens.

Another state of the art document BR 20 2012 0159266, filed on Feb. 27, 2012 by the same applicant of the present claim, discloses an arrangement applied to rigid endoscopes, comprising a disposable catheter longitudinally affixed on the rigid shaft of a conventional endoscope, receiving on its fore end a piece of equipment capable of releasing, in a controlled way, an amount of crystalloid solution, with flow directed by gravity, through the catheter channel, to the distal end, cleaning the objective lens of the endoscope during the video-surgery procedure.

In these devices disclosed by the state of the art documents, the saline or crystalloid solution application per se on the lens is not capable of making efficient cleaning of the endoscope's objective lens during surgical procedure, as the accumulation of saline on the lens may create an optical distortion condition, or in other condition, the accumulation of saline together with blood or body secretions may change the perfect visibility during the surgical procedure.

### Model Purpose

With the aim to solve the problems above, the present model has the purpose of providing means for lubrication and further removal of the saline or crystalloid solution from the lens during surgical procedure, in a non-invasive way.

### Model Proposal

it enables the surgeon to clean the objective lens without removing the instrument from the patient, and thus without interrupting the operation in progress. The surgeon is able to maintain the equipment tap partially open, in a continuous and controlled way, to generate constant flow of solution in contact with the lens. This causes the development of a crystalloid film in front of the optical pieces, automatically cleaning any type of secretion occasionally reaching them.

Also, upon the surgery completion, the physician just discard the catheter by removing it from the endoscope shaft, to enable its sterilization without any complications.

This new arrangement, object of protection of the present claim, can be coupled to any conventional surgical endoscope, providing a connector that enables its coupling to a saline equipment, arranged and affixed externally to the endoscope shaft by applying sterile adhesive tape.

### DESCRIPTION OF THE FIGURES

The abovementioned arrangement for cleaning the endoscope during video-surgery, object of protection of the present claim, will be explained in details by using the figures attached herein, where:

FIG. 1 shows a side view of the application of device for cleaning of the objective lens in a rigid endoscope;

FIG. 2 shows a magnified detail in perspective of the distal end and the fore end of the endoscope in a first constructive version;

FIG. 3 shows a magnified detail in perspective of the distal end and the fore end of the endoscope in a second constructive version;

### DETAILED DESCRIPTION OF THE MODEL

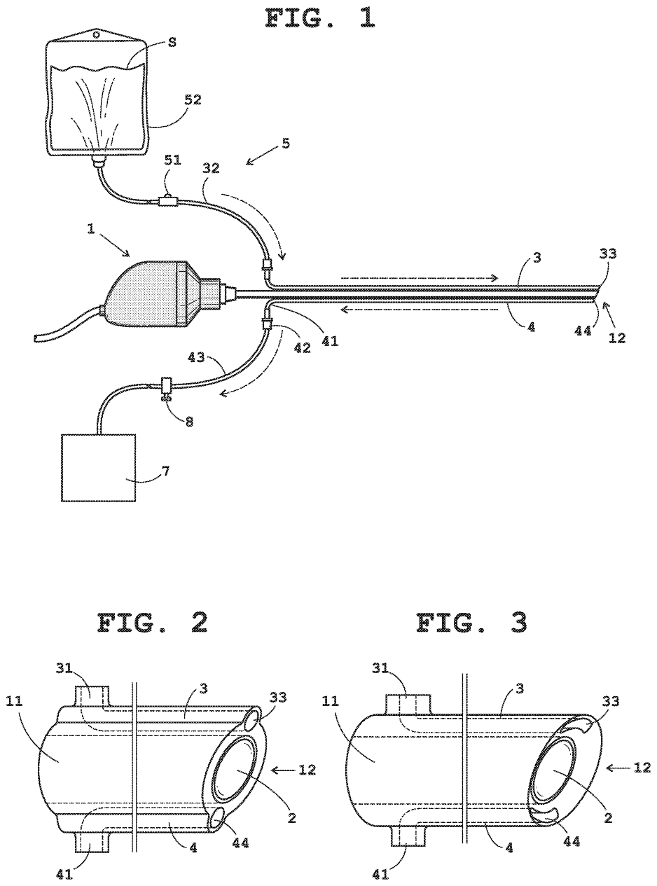

As shown in FIG. 1, the new arrangement applied on rigid endoscope (1) for cleaning of the objective lens (2), is comprised by two independent longitudinal channels developed along the rigid shaft (11) of the endoscope (1), with one channel (3) arranged on the upper part and the other channel (4) arranged on the lower part, both of them with access to the distal end of the endoscope (1).

The distal end (12) of the endoscope (1) is developed in acute angle in order that the upper channel (3) configure a more advanced access (33) and the lower channel (4) configure a backed off access (44) in relation to the objective lens (2).

The upper channel (3) has on its proximal end an upper access (31) where it is affixed a connector coupled to a hose (32) of a saline equipment (5), containing flow control tap (51), which is capable of releasing, in a controlled way, an amount of crystalloid solution that is directed by gravity through the saline bag (52) to the distal end (12) of the endoscope (1), lubricating the objective lens (2).

The lower channel (4) has in its proximal end a lower access (41) where it is fixed a connector (42), coupled to a hose (43), provided with activation valve (8), and that is connected to a suction device (7), which when actuated sucks the saline (S) from the distal end (12) of the endoscope (1), previously released by the access (33) of the upper channel (3), removing blood or body secretions, and consequently cleaning the lens (2) during video-surgery procedure.

1) “CONSTRUCTIVE ARRANGEMENT APPLIED IN RIGID ENDOSCOPE FOR CLEANING OF OBJECTIVE LENS DURING VIDEO-SURGICAL PROCEDURE”, aimed to clean the objective lens (2) of rigid endoscopes (1) during video-surgery procedure, with the rigid shaft (11) characterized to provide two independent longitudinal channels, one of them (3) arranged on the upper part, and the other (4) arranged on the lower part, both of them with access to the distal end of the endoscope (1) where is located the objective lens (2), and the upper channel (3) provides advanced access (33) and the lower channel (4) provides backed off access (44) in relation to the objective lens (2); the upper channel (3) provides on its proximal end a upper access (31) to a saline equipment (5) and the lower channel (4) has in its proximal end an access (41) where is fixed a connector (42) coupled to a hose (43), provided with activation valve (8), connected to suction device (7).
